(19) United States
(12) Patent Application Publication
(10) Pub. No.: US 2010/0178661 A1
(43) Pub. Date: Jul. 15, 2010

(54) AUTOMATIC TISSUE MICROARRAY APPARATUS AND METHOD FOR ARRAYING TISSUE USING THE SAME

(75) Inventors: Young Do Lee, Seoul (KR); Hyeong Jae Jeong, Seoul (KR)

Correspondence Address: HOLME ROBERTS & OWEN LLP, 1700 LINCOLN STREET, SUITE 4100, DENVER, CO, 80203, US

(73) Assignee: UNITMA CO., LTD., Seoul (KR)

(21) Appl. No.: 12/513,102

(22) PCT Filed: Oct. 15, 2007
(86) PCT No.: PCT/KR07/05025

§ 371 Date: Mar. 5, 2010

(30) Foreign Application Priority Data
Sep. 14, 2007 (KR) 10-2007-0093548

(51) Int. Cl. C12Q 1/02; C12M 1/36

(52) U.S. Cl. 435/29; 435/286.2

(57) ABSTRACT

Disclosed is an automatic tissue microarray apparatus that includes: a sample module being drivable and providing a space for mounting at least one donor block and at least one recipient block; an extract module being drivable in at least two directions and having at least one punching tip for punching a tissue from the at least one donor block and arraying the punched tissue in the at least one recipient block; and a controller for controlling the driving operations of the sample module and the extract module and, in response to an externally applied command, the punching and arraying operations of the extract module. The present invention securely provides convenience in use with improved work efficiency and contributes to precision of punching and arraying operations.

### TECHNICAL FIELD

The present invention relates to an automatic tissue micro-array apparatus and a method for preparation thereof that involves punching and arraying tissue samples, and more particularly, to an automatic tissue microarray apparatus and a method for preparation thereof that is automated to punch tissue from a donor block and array the punched tissue in a recipient block.

### BACKGROUND ART

The tissue biopsy (for research) in the field of medicine and biotechnology involves cutting a paraffin block of tissue in a microtome into very thin segments (4 to 8 μm thick) and mounting the tissue segments on a glass slide for microscopic examination. Only one tissue in the paraffin block is available on a single glass slide because its size is greater than 1 cm×2 cm×0.4 cm. Therefore the conventional method that allows only one tissue to be mounted on the glass slide requires a lot of time and cost in comparative analysis of multiple tissues, in consideration of a plurality of glass slides used, other consumables used for cutting the tissue, reagents for test and so forth. Moreover, the necessity of individual analyses of multiple tissues for comparative examination makes examinations inconsistent with lowered reliability. The technique of tissue micro-array that allows simultaneous analysis of multiple tissues is developed to deal with these problems.

The tissue micro-array technique refers to a material for research and its preparation method that involves mounting multiple tissues on a glass slide.

The biological tissues are human tissue, test animal tissue and cultured cells, and the glass slide is used for analysis of intercellular proteins, DNA or RNA and observation with microscopes. The slide may be applied to a wide range of examination techniques using the tissues, such as immunohistochemistry, in-situ hybridization, special stain, in-situ PCR and so forth.

A conventional method related to this tissue microarray technique involves a removal of tissues by manual punching with a punch and an array of the punched tissues in recipient blocks. However, such a conventional method features manual works done by the worker's hand and checked with eyes and thus causes many problems in the aspect of work efficiency and accuracy.

Another conventional method is disclosed in U.S. Pat. No. 6,383,801, which involves using a donor punch and a recipient punch separately from each other and simultaneously preparing a recipient block and a tissue microarray block. However, this method requires the user to mount the donor punch separately from the recipient punch and to replace the punches whenever the hole size of the recipient block changes, thereby causing inconvenience in use with reduced work efficiency.

### DISCLOSURE OF INVENTION

It is therefore an object of the present invention to provide an automatic tissue microarray apparatus and a method for preparation thereof that resolves the above-mentioned problems with the prior art.

It is another object of the present invention to provide an automatic tissue microarray apparatus and a method for preparation thereof that involves punching and arraying tissues in an automated way.

It is still another object of the present invention to provide an automatic tissue microarray apparatus and a method for preparation thereof that enables the user to check on the punching and arraying operations in real time.

It is still another object of the present invention to provide an automatic tissue microarray apparatus and a method for preparation thereof that securely provides precision of the punching and arraying operations.

It is still another object of the present invention to provide an automatic tissue microarray apparatus and a method for preparation thereof that involves a preparation of tissue microarray blocks in different sizes.

It is still another object of the present invention to provide an automatic tissue microarray apparatus and a method for preparation thereof that involves a simultaneous preparation of tissue microarray blocks in different sizes.

It is still further another object of the present invention to provide an automatic tissue microarray apparatus and a method for preparation thereof that guarantees an ease of handling and convenience in use.

To achieve the objects of the present invention, there is provided an automatic tissue microarray apparatus that includes: a sample module being drivable and providing a space for mounting at least one donor block and at least one recipient block; an extract module being drivable in at least two directions and having at least one punching tip for punching a tissue from the at least one donor block and arraying the punched tissue in the at least one recipient block; and a controller for controlling the driving operations of the sample module and the extract module and, in response to an externally applied command, the punching and arraying operations of the extract module.

The automatic tissue microarray apparatus further includes a vision module being drivable in at least two directions and used for taking images of the punching and arraying operations of the extract module and measuring location or height of the at least one donor block and locations of holes of the at least one recipient block.

The automatic tissue microarray apparatus further includes a display for displaying the taken images and the measured locations in response to image signals and measurement signals received from the vision module and having a touch-screen function to input a command and set a location by a user's touch.

The donor block is plural in number and comes in the same or different types. The at least one recipient block is a paraffin material structure with a plurality of cylindrical holes having at least one hole size.

The sample module is drivable in a first direction, the extract module being drivable in a second direction perpendicular to the first direction and a third direction perpendicular to the first and second directions.

The sample module includes: a sample stage having a space for mounting the plural donor blocks and the at least one recipient block; a sample driving stage having the sample stage mounted thereon and being used for moving the sample stage to a specific location; and a sample driving unit having a driving motor for driving the driving stage in the first direction.

The extract module includes: an extract tool having at least one punching tip for the punching and arraying operations; and a location driving unit having at least two driving motors and moving the extract tool to a specific location by a driving in the second and third directions.

The extract tool includes a plurality of the punching tips each having a different punching size, the punching tips being fixed on one rotatable circular body into a microscope's lens-attached structure or a windmill sail structure.

The extract tool further includes: a rotation driving unit having at least one rotation motor for moving one of the plural punching tips as selected by a user in a punching direction by rotating the extract tool, the punching direction being the third direction perpendicular to the plane; and a push driving unit having at least one push motor for pushing the tissue punched by the punching tip out of the punching tip.

The punching tip punches the tissue from the donor block by a descent of the location driving unit in the third direction and uses a probe provided therein and driven by the push driving unit to array the punched tissue in a specific hole of the recipient block.

The plural punching tips are provided as many as hole sizes of the recipient block mountable on the sample module.

The vision module includes: a displacement sensor for measuring the height of the donor block to adjust a punching depth; and a camera for taking images of the sample module and the punching and arraying operations of the extract module.

The vision module is driven integrally with or separately from the extract module.

The vision module further includes: a vision light being adjustable in light brightness for enhancing image quality of the camera; and at least one lens for adjusting image focus of the camera.

Determining punching location of the donor block, selecting holes of the recipient block and choosing one of the plural punching tips are performed by a command input through the touch-screen function.

In another embodiment of the present invention, there is provided a method for preparing an automatic tissue microarray block that includes: mounting at least one donor block and at least one recipient block on a sample module; determining the punching location of the at least one donor block, selecting holes of the at least one recipient block and choosing one of the plural punching tips having a punching size as large as the hole size of the at least one recipient block; and driving the selected punching tip to punch a tissue from the donor block and automatically arraying the punched tissue in the holes of the recipient block.

The recipient blocks, when plural in number, have the same or different hole sizes.

The method further includes measuring the height of the donor block with a displacement sensor to adjust a punching depth, before performing a punching operation with the selected punching tip.

Determining punching location of the at least one donor block, selecting holes of the recipient block and choosing one of the plural punching tips having a punching size as large as the hole size of the recipient block are performed by a command input through a touch screen.

### BRIEF DESCRIPTION OF DRAWINGS

FIG. 1 is a block diagram showing the construction of an automatic tissue microarray apparatus according to an embodiment of the present invention;

FIG. 2 is a schematic of the automatic tissue microarray apparatus shown in FIG. 1;

FIGS. 3 and 4 are illustrations showing the concrete construction and operation of the sample stage of FIG. 2;

FIG. 5 illustrates the status of the sample module completely set for punching and arraying a tissue;

FIGS. 6 and 7 are illustrations showing the concrete construction and operation of the extract module of FIG. 2;

FIGS. 8 and 9 are illustrations showing the concrete construction and operation of the vision module of FIG. 2;

FIG. 10 illustrates the whole operation of the automatic tissue microarray apparatus of FIG. 1; and

FIG. 11 is an external view of FIG. 2.

### BEST MODE FOR CARRYING OUT THE INVENTION

Hereinafter, the preferred embodiments of the present invention will be described in detail with reference to the accompanying drawings, which are intended for purpose of illustration only and are not intended to limit the scope of the invention.

The term “punching” as used herein means removing tissue and implies an operation of removing tissue with a punching tip. Accordingly, this term may be substituted by another appropriate term in describing tools or units for removing tissue other than the punching tip and may not used to limit or confine such tissue-removing tools or devices.

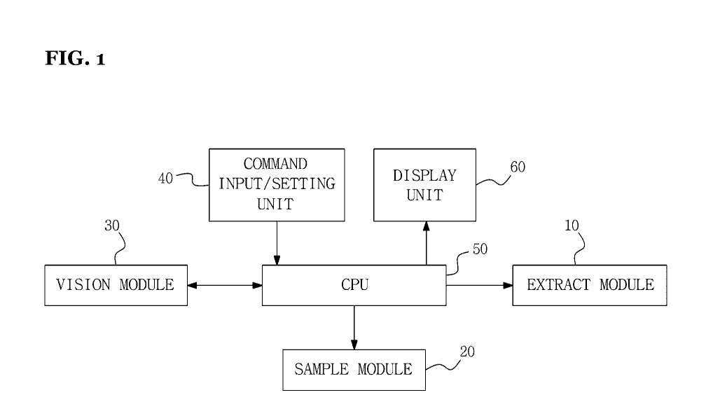

FIG. 1 is a block diagram of an automatic tissue microarray apparatus according to an embodiment of the present invention.

The automatic tissue microarray apparatus according to an embodiment of the present invention includes, as shown in FIG. 1, a sample module 20, an extract module 10, and a CPU 50. The apparatus may further include a vision module 30, a display unit 60 and a command input/setting unit 40. When the display unit 60 has a touch-screen function, the command input/setting unit 40 may be a component part of the display unit 60.

The sample module 20, drivable in a first direction (e.g., X-axis direction), provides a space for mounting at least one donor block and at least one recipient block.

The extract module 10, drivable in at least two directions (e.g., X- and Y-directions), has at least one punching tip to punch (remove) tissue from the at least one donor block and to array the punched tissue in the recipient block.

The CPU 50 controls the driving operations of the sample module 20 and the extract module 10 and, in response to an externally applied command, the punching and arraying operations of the extract module 10. For this, the CPU 50 has an embedded program for controlling the operations of the extract module 10 and the sample module 20. To enable the touch-screen function of the display unit 60, the CPU 50 is also constructed to control the operations in response to a touch-screen-based command input. That is, the CPU 50 may have a construction appropriate to the program-controlled system based on the touch screen.

The vision module 30 is drivable in at least two directions (for example, X- and Y-directions) and used for taking images of the punching and arraying operations of the extract module 10 and measuring the location or height of the donor block and the locations of the holes of the at least one recipient block.

The display unit 60 displays the taken images and the measured locations in response to the image signals and the measurement signals received from the vision module 30. Any well-known LCD monitor or the like may be used as the display unit 60. With a touch-screen function, the display unit 60 may be constructed to enable a command input and a location setting by the user's touch. In this case, the display unit 60 may substitute the command input/setting unit 40.

The command input/setting unit 40, although generally provided when the display unit 60 does not have a touch-screen function, may be used separately irrespective of whether the display unit 60 has a touch-screen function or not. The command input/setting unit 40 is used to input a command or set a location through different input devices such as keypad, mouse, keyboard, or the like.

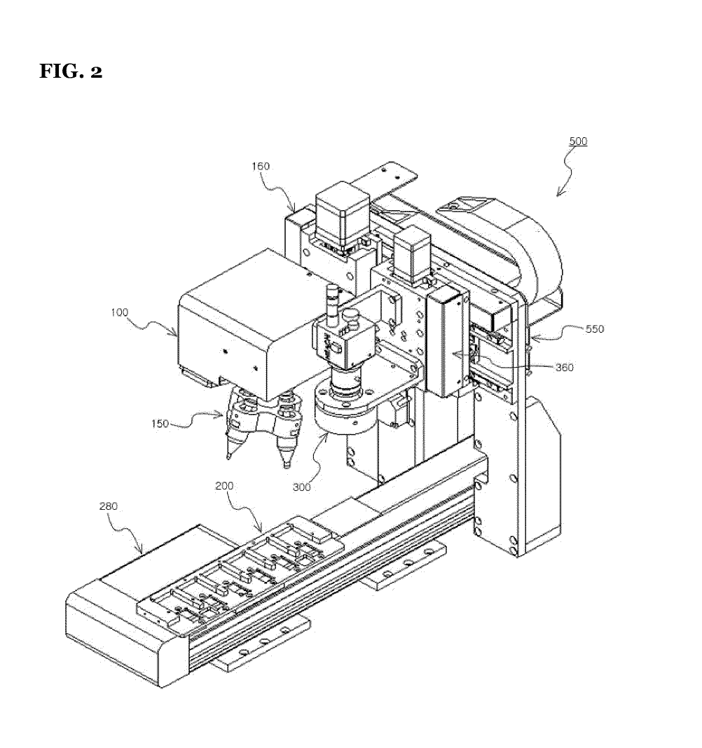

The component-specific operations and constructions of the above-constructed automatic tissue microarray apparatus are described below in detail with reference to FIGS. 2 through 10, which illustrate the constructions of the embodied apparatus unlike the block diagram of FIG. 1 and present a different symbol for the same component.

FIG. 2 is a schematic of the automatic tissue microarray apparatus 500 having a construction of FIG. 1.

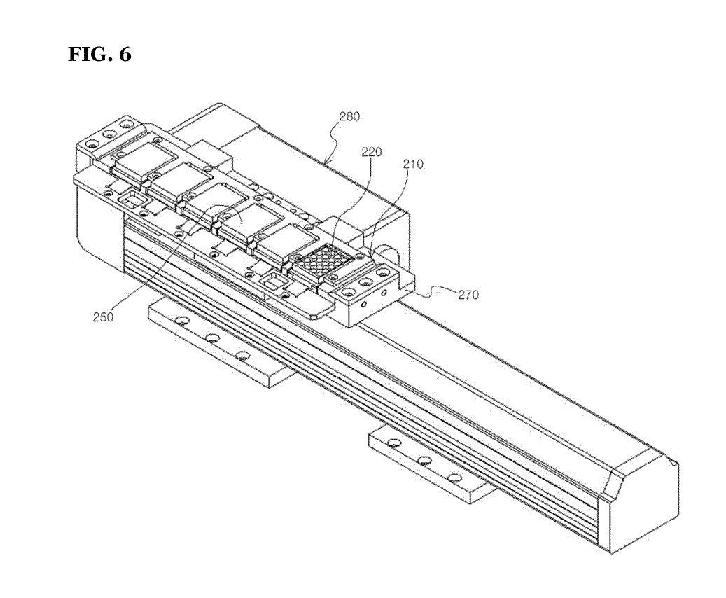

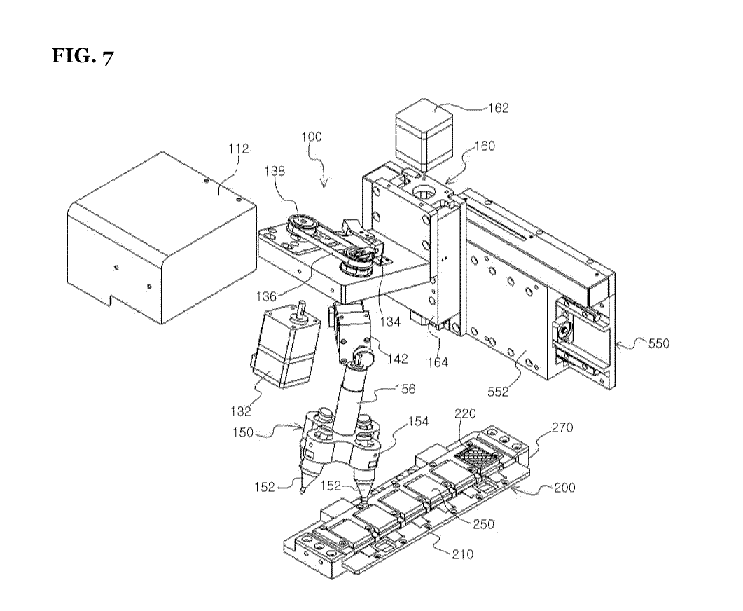

As illustrated in FIG. 2, the extract module 100 includes an extract tool 150 having at least one punching tip, and a location driving unit for driving the extract tool 150. The location driving unit for driving the extract tool 150 includes a second-direction (e.g., Y-axis direction) transfer stage 552 attached to the support 550, and a transfer robot 160 for driving the extract tool 150 in a third direction (e.g., Z-axis direction). The transfer robot 160 and the transfer stage 552 are constructed to move the entire of the extract module 100 as well as the extract tool 150. The extract module 100 is fixed to the support 550 through the transfer robot 160. The detailed construction and operation of the extract module 100 are described later in detail with reference to FIGS. 6 and 7.

The sample module 200 includes a sample stage 210, a sample driving stage 270, and a sample driving unit 280.

The sample stage 210 allows a space for mounting the at least one donor block and the at least one recipient block. The sample driving stage 270 has a structure drivable along the transfer path of the sample driving unit 280 and drives the sample stage 210 to be mounted.

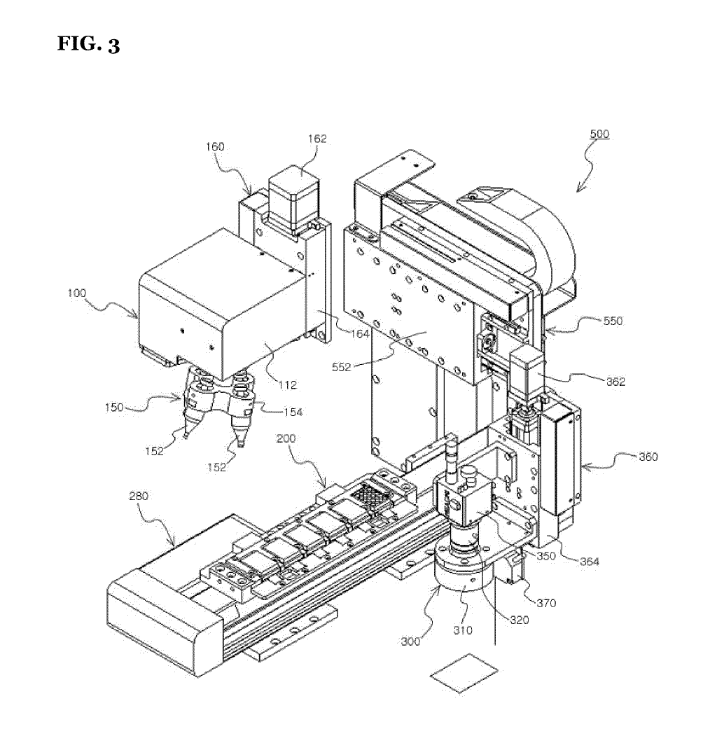

The sample driving unit 280 includes a transfer robot for driving the sample driving stage 270 in the first direction (e.g., X-axis direction). The CPU 50 controls the sample driving unit 280. The detailed construction and operation of the sample module 200 are described below in detail with reference to FIGS. 3, 4 and 5.

The vision module 300 measures the height of the donor block, takes images of the operations of the sample module 200 and the extract module 100 in real time, or provides images used for determined locations. The vision module 300 may include a camera (e.g., CCD camera), a displacement sensor, or the like, and additionally a transfer robot 360 as a location driving unit for second- and third-direction driving.

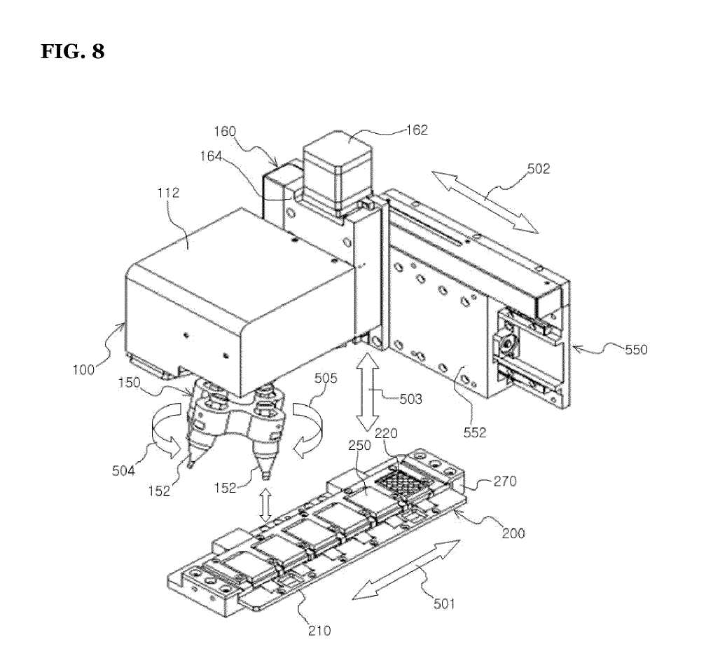

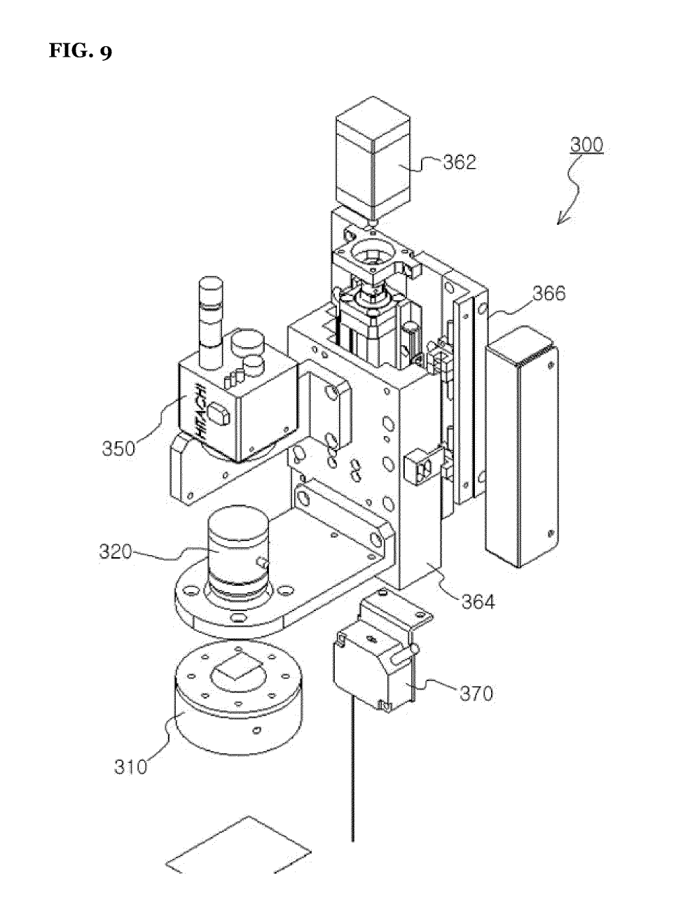

Here, the vision module 300 may be formed integrally with the extract module 100 and driven together with it. In this case, either the transfer robot 360 of the extract module 100 or the transfer robot 160 of the vision module 300 is omitted. Otherwise, when the vision module 300 is drivable separately from the extract module 100, both the transfer robot 360 of the extract module 100 and the transfer robot 160 of the vision module 300 are necessary. This embodiment exemplifies the case where the vision module 300 is drivable separately from the extract module 100. The detailed construction and operation of the vision module 300 are described below in detail with reference to FIGS. 8 and 9.

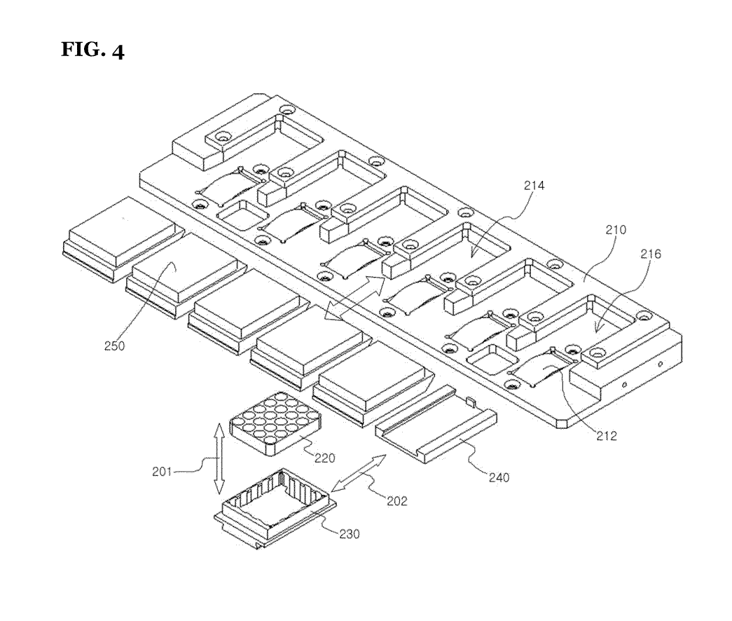

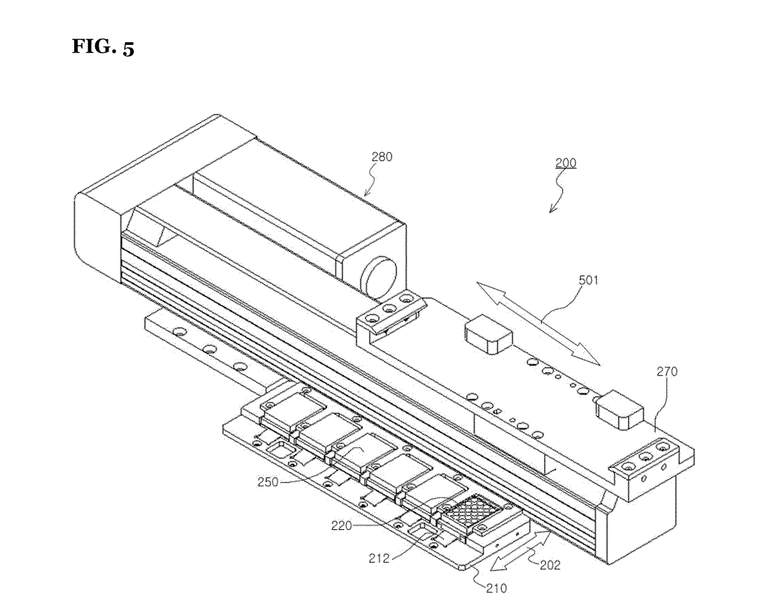

FIGS. 3 through 5 are perspectives showing the components of the sample module 200. FIG. 3 illustrates an operation of mounting a donor block 250 and a recipient block 220 on the sample stage 210, FIG. 4 illustrating an operation of mounting the sample stage 250 with the donor block 250 and the recipient block 220 on the sample driving stage 270, FIG. 5 illustrating the status of the sample module 200 completely set for punching and arraying tissue.

As shown in FIG. 3, the sample stage 210 has grooves 214 and 216 for mounting a predetermined number of donor blocks 250 and at least one recipient block 220. It is preferred that the sample stage 210 has many donor blocks 250, but the number of the donor blocks 250 depends on the size of the apparatus and may be appropriately determined. This embodiment exemplifies the sample stage 210 having five donor blocks 250.

The grooves 214 and 216 provided in the sample stage 210 are generally formed in a size corresponding to the size of the recipient block 220 or the donor block 250 mounted thereon. Aside from this, a plurality of sample stages 210 with grooves 214 and 216 of different sizes may be provided so as to mount the recipient block 220 or the donor block 250 that comes in different sizes.

The sample stage 210 may be plural in number with grooves of different sizes when the recipient block 220 or the donor block 250 has different lateral and vertical lengths. For mounting the recipient block 220 or the donor block 250 in this case, it is allowed to use the sample stage 210 with grooves that are a right size for the donor block or the recipient block.

The embodiment of the present invention exemplifies the case of using a plurality of donor blocks 250 (e.g., five donor blocks) and one recipient block 220. Cassette 230 for mounting the donor block 250 or the recipient block 220 and a cassette coupling unit 240 for coupling the cassette 230 to the grooves 214 and 216 of the sample stage 210 are prepared according to the standard. Accordingly, the donor block 250 and the recipient block 220 are required to meet the standard in lateral and vertical lengths.

The recipient block 220 as used herein is preferably the recipient block disclosed in PCT/KR2006/001298 filed by the applicant of the present invention. Alternatively, the recipient block 220 may include any types of recipient block having the same size of the recipient block disclosed in PCT/KR2006/001298. In other words, any donor block 250 or recipient block 220 is usable irrespective of type or thickness as long as it is a right size to be mounted on the cassette 230.

The recipient block 220 may have holes of the same or at least two different sizes.

The donor block 250 may include any types of biological tissue samples taken from the body of all animals including human, such as a piece of bone, surface of the skin and the like.

The donor block 250 is the same as the recipient block 220 in regard to its mounting procedure except for the mounting location. The operation of mounting the recipient block 220 on the sample stage is described as follows, while the operation of mounting the donor block 250 is omitted in the description.

First, the recipient block 220 is mounted on the cassette 230 in the arrow direction 201 of FIG. 3. The recipient block 220 as used herein is defined as a structure made of a paraffin material that has a plurality of cylindrical holes, but it may include an assembly of paraffin structure 220 with a plurality of cylindrical holes as mounted on the cassette 230. To avoid any confusion in the following description, the recipient block 220 is defined to a paraffin material having a plurality of cylindrical holes.

After the recipient block 220 is mounted on the cassette 230, the cassette 230 with the recipient block 220 is coupled to the cassette coupling unit 240 in the arrow direction 202 and mounted on the groove 216 of the sample stage 210. Here, the cassette 230 may be formed integrally with the cassette coupling unit 240.

Once coupled to the sample stage 210, the recipient block 220 becomes firmly fixed with a spring 212 of the sample stage 210. The mounting procedure of the donor block 250 is the same as that of the recipient block 220. The donor block 250 and the recipient block 220 are mounted on the sample stage 210 in the above-described procedure.

As illustrated in FIG. 4, the sample stage 210 with the recipient block 220 and the donor blocks 250 is mounted on the sample driving stage 270 of the sample driving unit 280. The sample driving stage 270 is formed integrally with the sample driving unit 280 but drivable in the first direction, namely, the arrow direction 501 along the transfer path provided in it. That is, the sample driving stage 270 is movable to a specific location in the first direction through a moving control means such as a motor (not shown) of the sample driving unit 280.

The reason why the sample driving stage 270 is formed separately from the sample stage 210 is because of the ease of mounting the recipient block 220 or the donor block 250. It is difficult to mount the recipient block 220 or the donor block 250 when the sample driving stage 270 is not separated from the sample stage 210. Such a separated construction is also intended to facilitate combination and detachment between a plurality of the sample stages 210 and the sample driving stage 270 when there are plural sample stages 210.

FIG. 5 shows a combined status of the sample stage 210 on the sample driving stage 270, that is, a complete set of the sample stage 210 and the sample driving stage 270. As illustrated in FIG. 5, the sample driving stage 270 is positioned on the transfer path of the sample driving unit 280, and the sample stage 210 is mounted on the sample driving stage 270. On the sample stage 270 are mounted a plurality of donor blocks 250 as a punching target and the recipient block 220 on which the punched tissue is to be arrayed.

The sample stage 210 is moved to a specific location either selectively or automatically, and the extract module 100 is operated to punch and array tissue samples.

In the following description, the sample module 200 may be illustrated without mentioning the sample driving unit 280 and/or the sample driving stage 270. This is to describe only the principal part of the sample module 200 and to avoid complexity of the drawings.

FIGS. 6 and 7 illustrate the detailed construction and operation of the extract module 100. FIG. 6 shows the detailed construction of the extract module 100, FIG. 7 showing the operation of the extract module 100.

As illustrated in FIG. 6, the extract module 100 includes a second-direction (e.g., Y-axis direction) transfer stage 552 attached the support 550 and a third-direction (e.g., Z-axis direction) transfer robot 160 for third-direction driving of an extract tool 150 as location driving units. The extract module 100 further includes the extract tool 150, and a rotation driving unit 132 and a push driving unit 142 as extract driving units for the punching and arraying operations of the extract tool 150. The extract module 100 still further includes additional power transmitting means (e.g., gear, belt, etc.) 138 and 136, and an extract tool case 112.

The extract module 100 is fixed on the support 550 through the transfer robot 160 that is a location driving unit. The transfer robot 160 and the transfer stage 552 are constructed to transfer the entire of the extract module 100 as well as the extract tool 150.

The transfer robot 160 has a transfer motor 162 for third-direction transfer and a transfer stage 164, and additionally a transfer motor (not shown) for second-direction transfer and a second-direction transfer robot (not shown). In another embodiment, the third-direction transfer robot 160 may additionally act as the second-direction transfer robot.

The extract tool 150 includes a plurality of punching tips 152, and a body 154 to which the punching tips 152 are attached. The rotation driving units 132, the push driving unit 152 and the extract tool 150 are associated with one another through a rotation axis 156.

The rotation driving unit 132 is driven to rotate the extract tool 150 and to select one of the plural punching tips 152 that has a predetermined punching size. The selected punching tip 152 is moved in the punching direction that is the third direction perpendicular to the plane. In other words, the rotation driving unit 132 is used to select one of the plural punching tips 152. The rotation driving unit 132 has at least one rotation motor.

The push driving unit 142 is used to push the tissue punched by the selected punching tip 152 out of the punching tip 152, that is, to array the punched tissue in the hole of the recipient block 220. For this operation, the push driving unit 142 has at least one push motor.

The punching tips 152 are plural in number with different punching sizes. The number of the punching tips 152 corresponds to that of the hole sizes of the recipient block 220. For example, four punching tips 152 are used when the recipient block 220 is available in four hole sizes. It is assumed in this embodiment that there are four hole sizes of the recipient block 220; 1 mm, 2 mm, 3 mm and 5 mm. Hence, four punching tips 152 having a punching size of 1 mm, 2 mm, 3 mm and 5 mm are employed.

Conventionally, it is required inconveniently to replace the punching tip whenever the hole size of the recipient block changes, because only one punching tip is provided for punching and arraying tissue samples. But the present invention resolves this inconvenient problem in regard to replacement of the punching tip.

The punching tips 152 are fixed on one rotatable circular body 154 into a microscope's lens-attached structure or a windmill sail structure. When one of the punching tips 152 fixed into a microscope's lens-attached structure or a windmill sail structure is selected by rotation of the circular body 154, the selected punching tip 152 is positioned in the third direction completely perpendicular to the sample module 200. The other punching tips 152 not selected are then positioned not to interfere with the selected punching tip performing a punching operation (that is, not to hit the donor block). The rotation driving unit 132 selects one of the punching tip 152 as described above.

The punching tip 152 includes a probe (not shown) in the form of a piston in the cylindrical inside of it. The punching tip 152 is constructed to move in the third direction (e.g., Z-axis direction) for punching a tissue and to array the punched tissue through the probe driven by the push driving unit 142. This operation is described in further detail as follows.

The probe has a structure extending to about 2 mm out of the punching tip 152 and retracting deep inside the punching tip 152 during the punching operation. In the punching operation, the probe retracts deep inside the punching tip 152 to fill the punching tip 152 with the punched tissue. After the completion of the punching operation, the probe is moved to 2 mm above a specific hole of the recipient block 220 to fill the hole of the recipient block 220 with the punched tissue. The probe is then operated to push the punched tissue out of the punching tip 152. Of course, the push driving unit 142 drives this operation of the probe.

The operation of the extract module 100 is described below with reference to FIG. 7 based on FIG. 6.

Referring to FIG. 7, the extract module 100 has a structure movable in the second and third directions 502 and 503. As previously described above, the sample stage 210 of the sample module 200 has a structure movable in the first direction 501. It is therefore possible to perform punching and arraying operations at a desired location by moving the sample stage 210 in the first direction 501 and the extract module 100 in the second and third directions 502 and 503.

The extract module 100 operates as follows. First, the extract module 100 is moved from the donor block 250 on the sample stage 210 to a location appropriate to perform a punching operation. This is realized by a transfer in the second and third directions 502 and 503 as driven by the transfer robot 160 and the transfer stage 552.

Subsequently, a selecting operation is performed to select one of the punching tips 152 that is a right size for the hole of the recipient block 220 on which the tissue sample is to be arrayed. This is realized by the rotating operation of the rotation driving unit 132. Here, the punching-tip selecting operation and the punching-location moving operation may be performed at the same time, or the punching-tip selecting operation may be performed prior to the punching-location moving operation.

The punching operation by the selected punching tip 152 follows the selecting operation. The punching operation is done by descending the punching tip 152 in the third direction using the transfer robot 160 that is a location driving unit for the third direction 503. The descending operation may continue until the punching tip 152 gets through the donor block 250 positioned under it. After descending in the third direction and passing through the donor block 250, the punching tip 250 ascends while the tissue removed from the donor block 250 is stuck in the punching tip 152.

To array the punched tissue, the punching tip 152 is required to move to the hole of the recipient block 220. When the punching tip 152 can move in the second direction 502 to reach the recipient block 220, the second-direction location driving unit 552 is used to drive the punching tip 152 to the arraying location on the recipient block 220. However, the sample stage 210 may move to make the punching tip 152 reach the recipient block 220 when the punching tip 152 moves in the first direction 501 from the punching location of the donor block 250 to the recipient block 220. In the present invention, the sample stage 210 moves in the first direction to make the punching tip 152 reach the arraying location. When a transfer in the third direction 503 is necessary, the transfer robot 160 that is a location driving unit for the third direction is used to move the punching tip 152 to the arraying location. The arraying location may be 2 mm right above the hole of the recipient block 220.

At the arraying location, the punching tip 152 driven by the push driving unit 142 is used to array the punched tissue in the hole of the recipient block 220.

With the holes in the recipient block 220 having different hole sizes, the punching tips corresponding to the selected hole sizes are sequentially chosen to perform the punching and arraying operations. When the recipient block 220 has holes of 1 mm and 3 mm, for example, a punching tip having a punching size of 1 mm is selected for the holes of 1 mm to perform the punching and arraying operations, and a punching tip having a punching size of 3 mm is selected for the holes of 3 mm.

FIGS. 8 and 9 illustrate the detailed construction and operation of the vision module 300. FIG. 8 shows the detailed construction of the vision module 300, FIG. 9 showing the operation of the vision module 300.

As illustrated in FIG. 8, the vision module 300 includes a camera 350 and a displacement sensor 370. The vision module 300 may further include a vision light 310 for adjusting the light brightness to enhance the image quality of the camera 350, and at least one lens 320 for adjusting the image focus of the camera 350. The vision module 300 may also include a transfer robot 360 that is a location driving unit for driving the vision module 300.

The camera 350 is to take images of the sample module 200 and the punching and arraying operations of the extract module 100. Additionally, the images taken by the camera 350 can be used in determining the punching location of the donor block 250, selecting the holes of the recipient block 220, and choosing one of plural punching tips 152. That is, the camera 350 is used not only to take images of the punching/arraying operation using the punching tip 152 but also to realize the touch-screen function.

For example, the user may touch a desired one of the donor blocks 250 on the display screen showing the images taken by the camera 350 to select the desired donor block 250. In the same manner, this touch-screen procedure is also applicable to the case of selecting the hole of the recipient block or one of the punching tips 152 having a desired punching size. The images taken by the camera 350 are recorded on a separate memory.

The displacement sensor 370 is to measure the height of the donor block 250 in order to adjust the punching depth. The displacement sensor 370 may include a measurement probe (not shown) of which the length changes according to the height of the donor block 250, in which case the height of the donor block 250 can be measured from the length variation of the measurement probe. In addition, the height measurement of the donor block 250 through the displacement sensor 370 includes a laser-based distance measurement method or any other measurement methods well known to those skilled in the art. It is obvious that the type of the displacement sensor 370 used depends on the measurement method using the displacement sensor 370.

The donor block 250 may include a wide range of tissues from skin tissue to bone chip as described above. The third-direction size (i.e., thickness) may not be all the same for every donor block 250 even though the first- and second-direction sizes are constant. Hence, measuring the thickness of the donor block 250 in advance allows adjustment of the punching depth in the punching operation of the punching tip 152. The thickness of the donor block 250 is a factor in determining the descending location of the punching tip 152, that is, how far the punching tip 152 must move down in the third direction.

The displacement sensor 370 includes different types of displacement sensors well known to those skilled in the art.

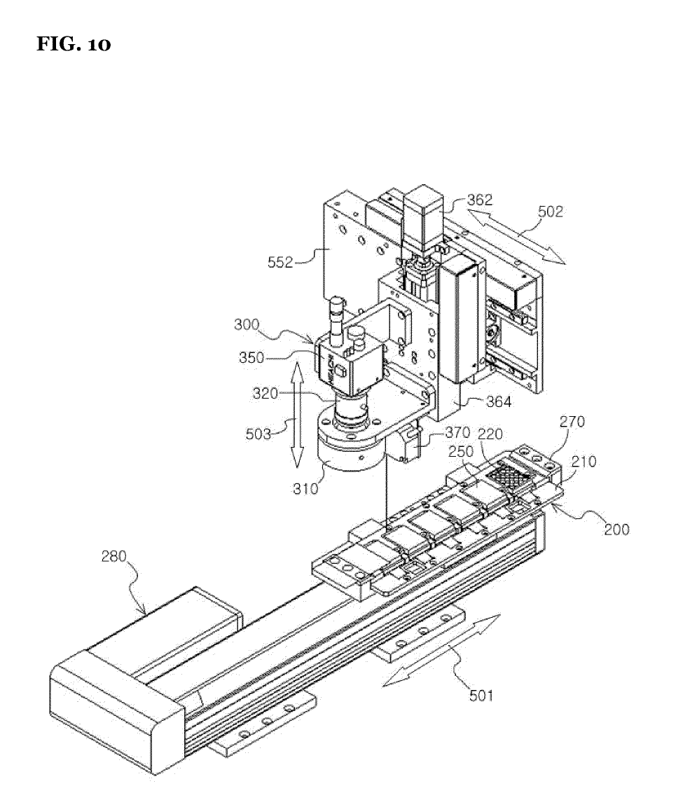

The vision module 300 may be driven integrally with or separately from the extract module 100, as previously mentioned in regard to the extract module 100. When driven separately from the extract module 100, the vision module 300 may additionally include a transfer robot (denoted by the reference number 360 in FIG. 10) for a third-direction driving.

The transfer robot 360 includes a transfer motor 362 for a transfer in the third direction, a transfer stage 364, and a support 366. If not illustrated, the vision module 300 is moved in the second direction in the same manner as the extract module 100, because the second-direction transfer stage 552 is shared between the vision module 300 and the extract module 100.

The operation of the vision module 300 is described below with reference to FIG. 9 based on FIG. 8.

The vision module 300 has a structure movable in the second and third directions 502 and 503. As already mentioned, the sample stage 210 of the sample module 100 is constructed to move in the first direction 501. It is thus possible to perform photographing and sensing at a desired location by moving the sample stage 210 in the first direction 501 and the extract module 100 in the second and third directions 502 and 503.

Before the punching operation using the extract module 100, the displacement sensor 370 is used to measure the height of the donor block 250. For this, the sample stage 210 is moved in the first direction 501, the displacement sensor 370 being moved in the second and third directions 502 and 503.

The camera 350 is moved in the second and third directions 502 and 503 to reach a desired location for taking images in real time. For example, the camera 350 takes images of the donor block 250 and the punching tip 152 in the punching step, and the recipient block 220 and the punching tip 152 in the arraying step. Therefore, the user can check on the punching/arraying operation through the display of the taken images.

FIG. 10 is an entire schematic of the automatic tissue microarray apparatus according to an embodiment of the present invention, showing the donor block 250 and the recipient block 220 completely set on the sample module 200, in relation to FIG. 2. The entire operation of the automatic tissue microarray apparatus is described below with reference to FIG. 10 on the basis of FIGS. 1 through 9.

As illustrated in FIG. 10, the donor block 250 and the recipient block 220 are mounted on the sample stage 210 of the sample module 200. The sample stage 210 is fixed on the sample driving stage 270, which is manually done by the user.

The user selects the type of the recipient block 220, that is, the mounted recipient block 220 through the display screen. That means selecting the hole size of the recipient block 220. Then the user selects one of the punching tip 152 that corresponds to the hole size of the recipient block 220. Selection of the punching tip 152 may be automated according to the selection of the type (i.e., hole size) of the recipient block 220.

The user determines a punching location of the donor block 250 and then the hole location of the recipient block 220 using the touch-screen function of the display screen.

Instead of the touch-screen function, any other command input means such as mouse, keyboard, keypad or the like may be used in selecting the hole of the recipient block 220, the punching location of the donor block 250 and the punching tip 152. For the user's quick selection, the command input means has quick buttons for immediately selecting the location of the donor block 250 or the hole size of the recipient block 220. For example, when the numeral key “1” is chosen on a keyboard, a command is automatically entered to set both the hole size of the recipient block 220 and the punching size of the punching tip 152 to 1 mm. This allows the user to select the hole size and the punching tip 152 immediately through the numeral key “1”. The data for the locations of the donor blocks 250 and the hole locations of the recipient block 220 are recorded on a separate memory.

Subsequently, the displacement sensor 370 is used to measure the height of the donor block 250. The height measurements for the donor block 250 are automatically recorded.

The operational mode enters the AUTO mode. The program stored in the CPU 50 executes the operation of punching tissue from the donor block 250 and the operation of arraying the punched tissue in the recipient block 220 in an automated way. The CPU 50 controls the arraying operation by automatically changing the holes of the recipient block 220 in order.

Instead of the AUTO mode, the operational mode may turn to the manual mode, in which the user checks the location of the punching tip 152 and manually controls the transfer of the punching tip 152 in person to execute punching and arraying operations. The manual mode involves separately designating the hole location of the recipient block 220 to array the tissue.

According to the present invention, the sample module 100 is drivable in the first direction, the extract module 100 and the vision module 300 being drivable in the second and third directions. Such a structure is capable of performing punching, arraying and photographing/measurement operations at a specific location. In addition, the present invention also allows a structure having the sample module 200 fixed and the extract module 100 and the vision module 300 drivable in the first, second and third directions; a structure having the sample module 200 drivable in the second and third directions and the extract module 100 and the vision module 300 drivable in the first direction; or a structure having the sample module 200 drivable in the first direction, the extract module 100 drivable in the second and third directions and the vision module 300 drivable in the first, second and third directions. These structures are readily realized by those skilled in the art.

The components shown in the illustrations but omitted in description are well known and readily understood to those skilled in the art and may be included in the scope of the present invention.

The above-described embodiment features a plurality of donor blocks 250 and one recipient block 220 mounted on the sample module 200.

In another embodiment, a plurality of recipient modules 220 are mounted on the sample module 200. The recipient modules 220 may have the same or different hole sizes. In other words, there may be used different methods of mounting a plurality of the recipient blocks 220 on occasion when needed.

It is assumed that two recipient blocks 220 having different hole sizes of, for example, 1 mm and 5 mm are mounted on the sample module 200. In the following description, if not illustrated, the recipient block having a hole size of 1 mm is expediently referred to as “a first recipient block”, the recipient block having a hole size of 5 mm “a second recipient block”, the punching tip having a punching size of 1 mm “a first punching tip”, the punching tip having a punching size of 5 mm “a second punching tip”.

With the first and second recipient blocks mounted on the sample module 200, two punching tips are selected on the extract module 100; the first punching tip having a punching size of 1 mm and the second punching tip having a punching size of 5 mm, corresponding to the hole sizes of the first and second recipients.

The punching and arraying operations are then performed in the same manner as described above in the case where one recipient block 220 is mounted on the sample module 200. For example, the operations involve a first step of using the first punching tip to punch a tissue from the donor block 250 and array the punched tissue in the hole of the first recipient block, and a second step of using the second punching tip to punch a tissue from the donor block 250 and array the punched tissue in the hole of the second recipient block.

The first and second steps are performed at the same time or alternately. Otherwise, the one of the first and second operations is first performed and then the other follows. In the punching operation, the donor block used as a target of the first punching tip may be the same as or different from the donor block used as a target of the second punching tip.

This case allows a number of recipient blocks mounted, thus making it possible to prepare a plurality of automatic tissue microarray blocks of different hole sizes in a short time.

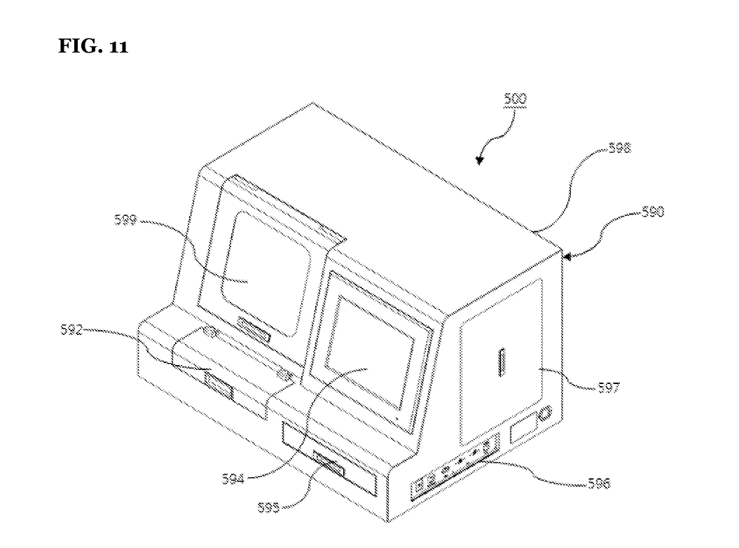

FIG. 11 shows an external view of the automatic tissue microarray apparatus according to the embodiment of the present invention.

As illustrated in FIG. 11, the automatic tissue microarray apparatus according to the embodiment of the present invention includes a rectangular box-shaped body 590, and maintenance panels 597 and 598 provided for maintenance on the top and lateral sides of the body 590. The apparatus further includes a door 592 for mounting the donor block 250 and the recipient block 220 on the sample module 200. Connection ports 596 are provided on the lateral side of the body 590 for connection to a keyboard, a mouse, external communications equipment, an external hard drive or the like. On the front side of the body 590 is formed a display screen 594 adjacent to the door 592. The display screen 594 as a part of the display unit 60 of FIG. 1 has a touch-screen function allowing the user to input a command by touch and displays images taken by the camera 350 of the vision module 300.

### INDUSTRIAL AVAILABILITY

As described above, in relation to the prior art, the automatic tissue microarray apparatus according to the embodiment of the present invention does not need a step of forming holes in the recipient block. Using ready-made recipient blocks in the present invention saves a lot of time and effort taken in forming holes in the recipient block. Contrary to the prior art that requires the punching tip replaced whenever the hole size of the recipient block changes, the present invention has a plurality of embedded size-specific punching tips and thus securely provides convenience in use. Such a system that embeds a plurality of punching tips allows mounting a plurality of recipient blocks having different hole sizes in a simultaneous manner, and thereby realizes a preparation of multiple automatic tissue microarray blocks having different hole sizes in a short time.

Moreover, the present invention includes a built-in controller using a touch-screen function to enhance convenience in use and ease of control, while the conventional equipment inconveniently employs an external control system using external PC connections or the like.

While this invention has been described in connection with the embodiments, it is to be understood to those skilled in the art that the invention is not limited to the disclosed embodiments, but is intended to cover various modifications and equivalent variants.

What is claimed is:

1. An automatic tissue microarray apparatus comprising:
a sample module being drivable and having a space formed therein for mounting at least one donor block and at least one recipient block;
an extract module being drivable in at least two directions and having at least one punching tip for punching a tissue from the donor block and arraying a punched tissue in the recipient block; and
a controller for controlling driving operations of the sample module and the extract module and, in response to an externally applied command, a punching and arraying operation of the extract module.

2. The automatic tissue microarray apparatus as claimed in claim 1, further comprising:
a vision module being drivable in at least two directions and used for taking images of the punching and arraying operations of the extract module and a measuring location or height of the donor block and locations of at least one hole of the recipient block.

3. The automatic tissue microarray apparatus as claimed in claim 2, further comprising:
a display for displaying the images and the measuring location in response to image signals and measurement signals received from the vision module and having a touch-screen function to input a command and set a location by a user's touch.

4. The automatic tissue microarray apparatus as claimed in claim 1, wherein the donor block is plural in number and comes in the same or different types.

5. The automatic tissue microarray apparatus as claimed in claim 4, wherein the recipient block is a paraffin material structure with a plurality of cylindrical holes having at least one hole size.

6. The automatic tissue microarray apparatus as claimed in claim 1, wherein the sample module is drivable in a first direction, the extract module being drivable in a second direction perpendicular to the first direction and a third direction perpendicular to the first and second directions.

7. The automatic tissue microarray apparatus as claimed in claim 5, wherein the sample module comprises:
a sample stage having a space for mounting a plurality of donor blocks and the recipient block;
a sample driving stage having the sample stage mounted thereon and being used for moving the sample stage to a specific location; and
a sample driving unit having a driving motor for driving the sample driving stage in a first direction.

8. The automatic tissue microarray apparatus as claimed in claim 1, wherein the extract module comprises:
an extracting tool having at least one punching tip for punching and arraying operations; and
a location driving unit having at least two driving motors and moving the extract tool to a specific location by a driving in a second and a third direction.

9. The automatic tissue microarray apparatus as claimed in claim 8, wherein the extracting tool comprises a plurality of punching tips each having a different punching size, the punching tips being fixed on one rotatable circular body into a microscope's lens-attached structure or a windmill sail structure.

10. The automatic tissue microarray apparatus as claimed in claim 9, wherein the extracting tool further comprises:
a rotation driving unit having at least one rotation motor for moving one of the plurality of punching tips as selected by a user in a punching direction by rotating the extract tool, the punching direction being the third direction perpendicular to a plane; and
a push driving unit having at least one push motor for pushing the tissue punched by the punching tip out of the punching tip.

11. The automatic tissue microarray apparatus as claimed in claim 10, wherein the punching tip punches the tissue from the donor block by a descent of the location driving unit in the third direction and has a probe provided therein and driven by the push driving unit to array the punched tissue in a specific hole of the recipient block.

12. The automatic tissue microarray apparatus as claimed in claim 11, wherein the plurality of punching tips are provided as many as hole sizes of the recipient block mountable on the sample module.

13. The automatic tissue microarray apparatus as claimed in claim 2, wherein the vision module comprises:
a displacement sensor for measuring the height of the donor block to adjust a punching depth; and
a camera for taking images of the sample module and the punching and arraying operations of the extract module.

14. The automatic tissue microarray apparatus as claimed in claim 13, wherein the vision module is driven integrally with or separately from the extract module.

15. The automatic tissue microarray apparatus as claimed in claim 13, wherein the vision module further comprises:
a vision light being adjustable in light brightness for enhancing image quality of the camera; and
at least one lens for adjusting image focus of the camera.

16. The automatic tissue microarray apparatus as claimed in claim 3, wherein determining a punching location of the donor block, selecting holes of the recipient block and choosing at least one of the punching tips are performed by a command input through the touch-screen function.

17. A method for arraying automatically tissue samples comprising:
mounting the donor block and the recipient block on a sample module;
determining a punching location of the donor block, selecting at least one hole of the recipient block and choosing one of the plurality of punching tips having a punching size as large as a hole size of the recipient block; and
driving the selected punching tip to punch a tissue from the donor block and automatically arraying a punched tissue in the holes of the recipient block.

18. The method as claimed in claim 17, wherein the recipient block has the same or different hole sizes when a plurality of recipient blocks are mounted.

19. The method as claimed in claim 17, further comprising:
measuring a height of the donor block with a displacement sensor to adjust a punching depth, before performing a punching operation with the selected punching tip.

20. The method as claimed in claim 17, wherein determining the punching location of the donor block, selecting holes of the recipient block and choosing one of the plurality of punching tips having a punching size as large as the hole size of the recipient block are performed by a command input through a touch screen.
